(19) United States
(12) Patent Application Publication
(10) Pub. No.: US 2021/0210204 A1
(43) Pub. Date: Jul. 8, 2021

(54) ASSOCIATING A PATIENT WITH A DEVICE

(71) Applicant: Hill-Rom Services, Inc., Batesville, IN (US)

(72) Inventor: Patrick J. Noffke, Hartland, WI (US)

(21) Appl. No.: 17/143,478

(22) Filed: Jan. 7, 2021

Related U.S. Application Data
(60) Provisional application No. 62/958,630, filed on Jan. 8, 2020.

(51) Int. Cl. G16H 40/67; G16H 10/60; G06F 3/01; G08B 21/02

(52) U.S. Cl. CPC G16H 40/67; G16H 10/60; G08B 21/0247; G08B 21/0211; G06F 3/011

(57) ABSTRACT

This disclosure is directed towards a patient management system for associating a patient with a device based on sensed inputs. A computing device of a patient management system may receive, from a device coupled to a patient, a first signal associated with a physiological parameter, where the first signal has a first temporal component. Additionally, the patient management system may receive, from a healthcare establishment device, a second signal associated with the physiological parameter, where the second signal has a second temporal component. The patient management system may determine that the first temporal component aligns with the second temporal component. Based on this determination, the patient management system may associate the patient with the healthcare establishment device. The patient management system may output the activity score to an electronic device, such as a clinician device and/or a device associated with the patient.

### CROSS-REFERENCE TO RELATED APPLICATION

This application claims priority to U.S. Provisional Application No. 62/958,630, filed on Jan. 8, 2020 and entitled “Associating A Patient With A Device,” the entirety of which is incorporated herein by reference.

### TECHNICAL FIELD

This application is directed to a patient management system, and in particular, to a system configured to associate a patient with a device based on sensed physiological parameters.

### BACKGROUND

As technology continues to advance, healthcare establishment devices provide increased functionality to patients and caregivers. For example, a healthcare establishment device such as hospital bed may generate data about a patient based on sensed inputs, such as a position of the patient in the bed, activities of the patient on or near the bed, respiration rate of the patient sensed by the bed, heart rate of the patient sensed by the bed, and the like. However, ensuring that such activities are actually being performed by the patient (and not, for instance, a family member, a caregiver, a different patient, a cleaning service, and so forth) can often present challenges, which may result in inaccurate data being associated with the patient.

The various example embodiments of the present disclosure are directed toward overcoming one or more of the deficiencies associated with patient management systems.

### SUMMARY

Broadly, the systems and methods disclosed and contemplated herein are directed towards a patient management system for accurately associating a patient with a device based on sensed inputs. In some examples, a computing device of a patient management system may receive, from a device coupled to a patient, a first signal associated with a physiological parameter, where the first signal has a first temporal component. Additionally, the patient management system may receive, from a healthcare establishment device, a second signal associated with the physiological parameter, where the second signal has a second temporal component. The patient management system may determine that the first temporal component aligns with the second temporal component. Based on determining that the first temporal component aligns with the second temporal component, the patient management system may associate the patient with the healthcare establishment device. The patient management system may output the activity score to an electronic device, such as a clinician device and/or a device associated with the patient.

### BRIEF DESCRIPTION OF THE DRAWINGS

FIG. 1 shows a schematic block diagram of an example patient management system environment.

FIG. 2 shows an example of signals which may be used to determine whether to associate a patient with a healthcare establishment device.

FIG. 3 shows an example system configured to adjust and/or align signals based on a delay associated with at least one of the signals.

FIG. 4 shows a schematic block diagram of a signal association component that may utilize signal strength of signals in determining whether to associate a patient with a healthcare establishment device.

FIG. 5 is an example process for associating a patient with a healthcare establishment device based on whether signals that may be associated with the patient at least partially align, according to the techniques described herein.

FIG. 6 is an example computing system and device which may be used to implement the described techniques.

### DETAILED DESCRIPTION

Various embodiments of the present disclosure will be described in detail with reference to the drawings, wherein like reference numerals represent like parts and assemblies throughout the several views. Additionally, any examples set forth in this specification are not intended to be limiting and merely set forth some of the many possible embodiments.

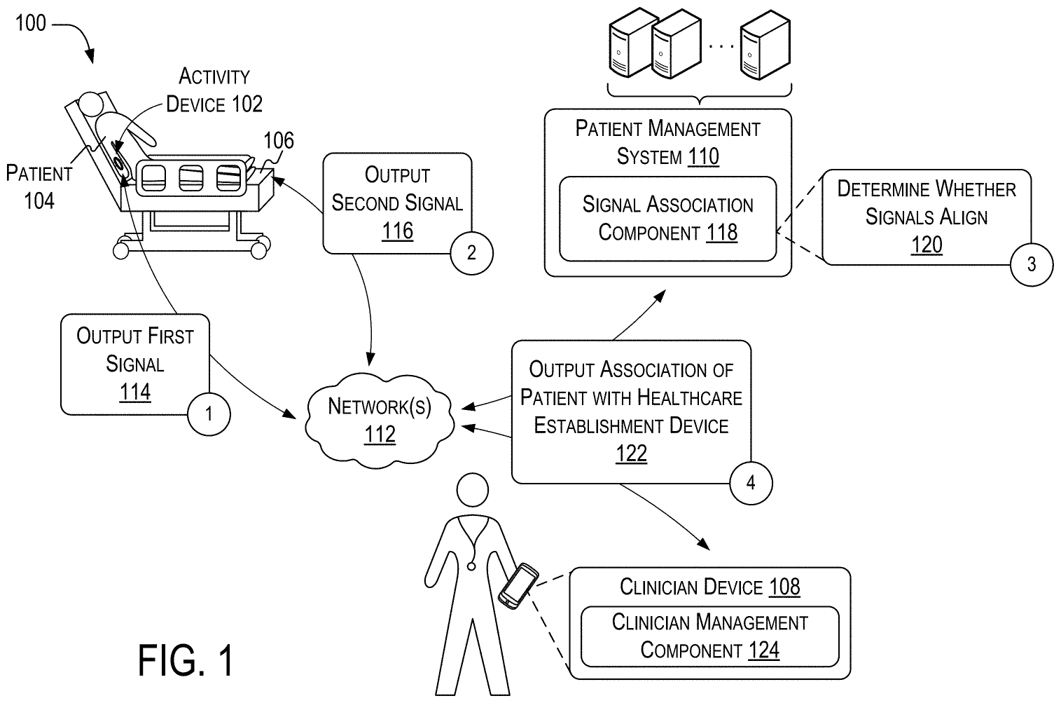

FIG. 1 shows a schematic block diagram of an example patient management system environment 100. The example patient management system environment 100 includes at least one activity device 102 (e.g., worn by a patient 104), a healthcare establishment device 106 (e.g., a hospital bed), a clinician device 108, and a patient management system 110. The activity device 102, the healthcare establishment device 106, the clinician device 108, and/or the patient management system 110 may be in communication via one or more networks 112.

In some examples, the activity device 102 may be any suitable portable computing device that can store data and be transported by the patient 104, such as a watch, a necklace, a ring, a bracelet, eyeglasses, shoe(s), clothing, a patch, a belt, a band, and/or other type of accessory. Examples are also contemplated in which the activity device 102 comprises a phone, tablet, laptop computer, or other computing device that may not necessarily be “worn” on the body of the patient 104. In some cases, the activity device 102 may include one or more sensors, such as a heart rate sensor, respiration sensor, glucose sensor, blood pressure sensor, diagnostic sensor, motion sensor (e.g., accelerometer, gyroscope, etc.), and so forth.

In some examples, the healthcare establishment device 106 may be one of multiple healthcare establishment devices that generally exist in a healthcare establishment (e.g., doctor's office, hospital, clinic, dentist's office, pharmacy, ambulance, and the like) that may impact and/or monitor the health of the patient 104. For instance, the healthcare establishment device 106 may include a blood pressure device, an SpO₂ device, a temperature device, a respiratory device, a bodyweight scale, an otoscope, an ophthalmoscope, a stethoscope, a vision screening device, a hearing screening device, a microscope, an ECG device, an overhead lift device, a pressure-sensitive mat device, a bed and/or other furniture, and so on.

In examples where the healthcare establishment device 106 is a hospital bed (or other type of furniture), the healthcare establishment device 106 may include load cells, air bladder pressure sensors, thermal sensors, capacitive sensors (e.g., to sense a ballistocardiogram signal of the patient 104), pressure mapping sensors, ultrasonic sensors (e.g., to determine a distance of the patient 104 and/or a healthcare provider from the hospital bed), motion sensors, and/or the like. Further, in examples where the healthcare establishment device 106 is a hospital bed (or other type of furniture), the healthcare establishment device 106 may generate articulation data corresponding to an angle of the head and/or feet of the bed, which the healthcare establishment device 106 may use to determine a position or posture of the patient 104. While the healthcare establishment device 106 is described as existing within a healthcare establishment, examples are considered in which such devices may be found outside of a healthcare establishment, in some cases.

In examples, the clinician device 108 may include a computing device such as a mobile phone, a tablet computer, a laptop computer, a desktop computer, and so forth which provides a clinician (e.g., a doctor, nurse, technician, pharmacist, dentist, etc.) with information about the health of the patient 104. In some cases, the clinician device 108 may exist within a healthcare establishment (e.g., alongside the healthcare establishment device 106), although examples are also considered in which the clinician device 108 exists and/or is transported outside of a healthcare establishment, such as a doctor's mobile phone or home desktop computer that the doctor may use when the doctor is on-call. Alternatively or additionally, the clinician device 108 may include a device used in emergency medical situations (e.g., in an ambulance and/or accessible by emergency medical technicians (EMTs)), where the clinician devices in these situations can add, remove, change, and/or otherwise access data stored on the activity device 102.

The activity device 102, the healthcare establishment device 106, and/or the clinician device 108 may include a processor, microprocessor, and/or other computing device components, shown and described below. For instance, the activity device 102, the healthcare establishment device 106, and/or the clinician device 108 may be configured as mobile phones, tablet computers, laptop computers, etc., to deliver or communicate patient data amongst one another and to other devices.

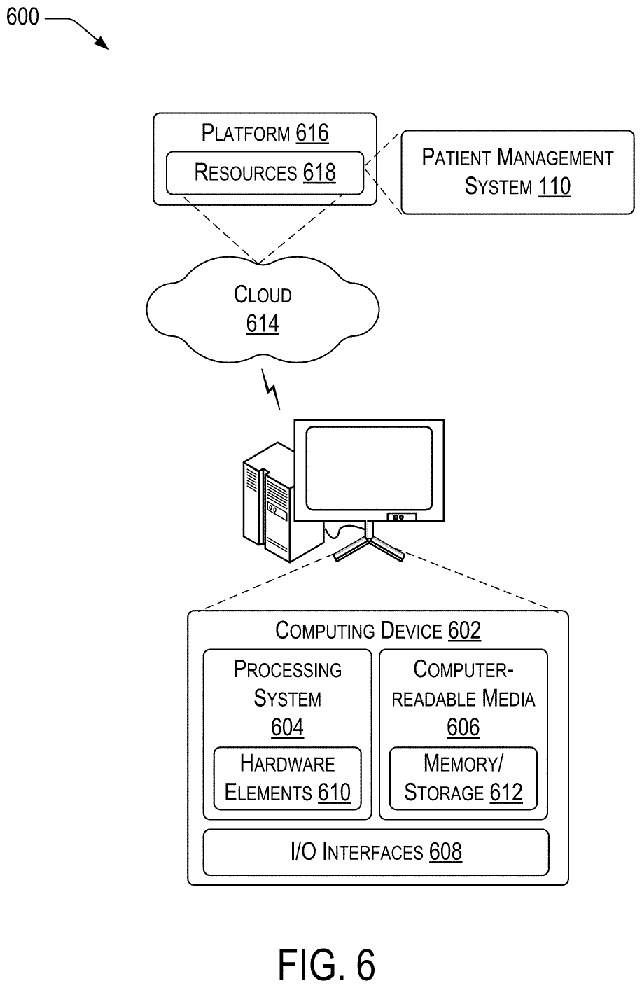

The patient management system 110 may be comprised of one or more server computing devices, which may communicate with the activity device 102, the healthcare establishment device 106, and/or the clinician device 108 to respond to queries, receive data, and so forth. Communication between the patient management system 110, the activity device 102, the healthcare establishment device 106, and/or the clinician device 108 occurs via the network 112, where the communication can include signals and/or patient data related to the health of the patient 104. A server of the patient management system 110 can act on these requests from the activity device 102, the healthcare establishment device 106, and/or the clinician device 108, determine one or more responses to those queries, and respond back to activity device 102, the healthcare establishment device 106, and/or the clinician device 108. A server of the patient management system 110 may also include one or more processors, microprocessors, or other computing devices as discussed in more detail in relation to FIG. 6.

The patient management system 110 may include one or more database systems accessible by a server storing different types of information. For instance, a database can store correlations and algorithms used to manage the signals and/or patient data to be shared between the activity device 102, the healthcare establishment device 106, and/or the clinician device 108. A database can also include clinical data. A database may reside on a server of the patient management system 110 or on separate computing device(s) accessible by the patient management system 110.

The network 112 is typically any type of wireless network or other communication network known in the art. Examples of the network 112 include the Internet, an intranet, a wide area network (WAN), a local area network (LAN), and a virtual private network (VPN), cellular network connections and connections made using protocols such as 802.11a, b, g, n and/or ac. Alternatively or additionally, the network 112 may include a nanoscale network, a near-field communication network, a body-area network (BAN), a personal-area network (PAN), a near-me area network (NAN), a campus-area network (CAN), and/or an inter-area network (IAN).

In some examples, the patient management system 110, the activity device 102, the healthcare establishment device 106, and/or the clinician device 108 may generate, store, and/or selectively share signals and/or patient data between one another to provide the patient 104 and/or clinicians treating the patient 104 with improved outcomes by accurately associating the patient 104 with the healthcare establishment device 106. For instance, the activity device 102 and/or the healthcare establishment device 106 may sense an activity associated with the patient 104, such as based on movement, heart rate, respiratory rate, blood pressure, SpO₂, and so forth, and store patient data 114 in the form of values associated with the activity for at least a period of time. The period of time may be a predetermined time (e.g., one minute, one hour, one day, etc.), or a variable time (e.g., between visits by a clinician to a hospital room of the patient, until stopped by the patient 104 and/or a clinician, etc.).

In some cases, an operation 114 (indicated by “1”) may include outputting, by the activity device 102, a first signal. For example, the first signal may correspond to values associated with a sensed activity of the patient. For instance, the first signal may correspond to an electrocardiogram (ECG) signal, a respiration signal, an impedance cardiogram signal, a photoplethysmogram (PPG) signal, an ultrasound signal, a motion signal, and so forth. The first signal may have a temporal component associated with the values included in the first signal, where the temporal component associated with an individual value identifies a time that the individual value occurred. Accordingly, the temporal component may be used to determine how the values in the first signal change as time progresses. In an illustrative example, the first signal may correspond to a respiratory rate of the patient 104 over time, as monitored by the activity device 102 coupled to the patient 104.

An operation 116 (indicated by “2”) may include outputting, by the healthcare establishment device 106, a second signal. Similar to the first signal, the second signal may correspond to values associated with a sensed activity of the patient. For instance, in an example where the healthcare establishment device 106 is a hospital bed, the signal may be a motion signal, a respiration signal, a ballistocardiogram signal, and/or a cardiac pulse signal generated by a load cell, a capacitive sensor, and/or a pressure sensor included in the hospital bed. The second signal may correspond to a same (or similar) activity as the activity monitored by the activity device 102 and associated with the first signal, and/or may be a different activity than the activity monitored by the activity device 102 and associated with the first signal.

The second signal may also have a temporal component associated with the values included in the second signal, where the temporal component associated with an individual value identifies a time that the individual value occurred. Accordingly, the temporal component may be used to determine how the values in the second signal change as time progresses. In examples, the temporal component associated with the second signal may be a same (or similar) temporal component as the temporal component associated with the first signal generated by the activity device 102, and/or may be a different temporal component than the temporal component associated with the first signal generated by the activity device 102. In an illustrative example, the second signal may correspond to a respiratory rate of the patient 104 over time, as monitored by a pressure sensor of the healthcare establishment device 106.

In some examples, the activity device 102 may output the first signal to a signal association component 118 of the patient management system 110. Additionally, in some cases, the healthcare establishment device 106 may output the second signal to the signal association component 118 as well. The signal association component 118 may be configured to determine whether the first signal and the second signal align, and if so, may be configured to associate the patient 104 with the healthcare establishment device 106 that generated the second signal.

For example, an operation 120 (indicated by “3”) may include determining whether the signals align. To do so, the signal association component 118 may detect events associated with a physiological parameter included in the signals (e.g., movement, breaths, pulse, etc.) and determine whether an event included in the first signal occurs within a threshold amount of time of an associated event included in the second signal. The threshold amount of time may be a predetermined amount of time, which may differ depending upon a type of signal and/or event being analyzed. For instance, a cardiac event may have a smaller threshold amount of time (e.g., 50 milliseconds, 100 milliseconds, 150 milliseconds, etc.) than a threshold amount of time associated with a respiratory event (e.g., 250 milliseconds, 500 milliseconds, 750 milliseconds, etc.) due to the cardiac event being shorter than the respiratory event, the cardiac event measurement being more precise than the respiratory event measurement, and the like.

In some cases, the signal association component 118 may account for transmission delays (or other signal delays) when determining whether the first signal aligns with the second signal. For instance, the signal association component 118 may shift the time component of the first signal such that the temporal component of the first signal matches (or is within a threshold amount) of the temporal component of the second signal. The signal association component 118 may use time stamps included in the first signal and the second signal to determine whether to alter either one of the signals such that the temporal components of the two signals generally coincide.

Alternatively or additionally, the signal association component 118 may associate the patient 104 with the healthcare establishment device 106 based at least in part on a signal strength of signals communicated between the activity device 102 and the healthcare establishment device 106. For instance, in addition to outputting the first signal to the patient management system 110 as described in relation to the operation 114, the activity device 102 may also output the first signal (and/or a different signal) to the healthcare establishment device 106. In some cases, other activity devices (e.g., worn by patients other than the patient 104) may output signals that are received by the healthcare establishment device 106 as well. The signal association component 118 may receive indications of signal strengths of signals communicated from the various activity devices, including the activity device 102, with the healthcare establishment device 106. The signal association component 118 may rank the signals communicated to and/or from the healthcare establishment device 106 with the various activity devices. The signal association component 118 may use the ranking of the signal strengths as a factor in determining whether to associate the patient 104 with the healthcare establishment device 106, either alternatively or in addition to aligning the first and second signals.

As mentioned above, the signal association component 118 may associate the patient 104 with the healthcare establishment device 106 using signals that indicate different types of events, and/or are different signal types. For instance, the activity device 102 may output a motion signal as the first signal that indicates a change in posture of the patient 104 (e.g., from laying to sitting). The healthcare establishment device 106 may be a hospital bed that outputs a pressure signal as the second signal associated with a location of weight of a person on the hospital bed changing. Even though the first signal and the second signal in this example are different signal types (e.g., motion and pressure), the signal association component 118 may associate the patient 104 with the healthcare establishment device 106 based on the change in posture of the patient 104 and the change in location of weight on the hospital bed being generally at a same time.

An operation 122 (indicated by “4”) may include outputting the association of the patient with the healthcare establishment device. For instance, the signal association component 118 may output an association of the patient 104 with the healthcare establishment device 106 to a clinician management component 124 of the clinician device 108. In some cases, the clinician management component 124 may display a verification message to a clinician associated with the clinician device 108 based on receiving the association from the signal association component 118. The clinician may verify that the patient 104 is associated with the healthcare establishment device 106 using the verification message, which may be performed remotely from the location of the healthcare establishment device 106 itself. This may relieve clinicians from conventional procedures of associating a patient with a healthcare establishment device, such as manually recording all hospital bed entries and exits, thus freeing the clinician to perform more essential tasks throughout the healthcare establishment, and enabling extended unsupervised monitoring by the physiological devices.

Additionally, in some examples, the signal association component 118 may output the association to the activity device 102 and/or the healthcare establishment device 106 as well. In some cases, the signal association component 118 may continuously (or semi-continuously) output the association, if present, to the activity device 102 and/or the healthcare establishment device 106. Based on the association, the activity device 102 and/or the healthcare establishment device 106 may more accurately determine whether detected activities or exercises are performed by the patient 104 or another person. For instance, consider a scenario in which the healthcare establishment device 106 (in this case a hospital bed) outputs a pressure signal indicating that a person is sitting on the bed, but the activity device 102 outputs a signal that the patient 104 is in a standing position. The healthcare establishment device 106 may use the association of the patient 104 with the hospital bed to determine that the patient 104 is not the person sitting on the bed, and thus may prevent an activity indication that corresponds to the patient 104 sitting from being output. Conventional systems that do not automatically associate a patient with a healthcare establishment device at regular intervals may record the activity of sitting on the bed as an activity performed by the patient, thus leading to inaccurate activity metrics. Therefore, the described techniques improve accuracy by appropriately associating the patient 104 with the healthcare establishment device 106 based on signals generated by the activity device 102 and the healthcare establishment device 106.

Although generally described in relation to associating a single patient with a healthcare establishment device, examples are considered in which the described techniques may be implemented to associate multiple patients with multiple healthcare establishment devices as well. For example, the signal association component 118 may receive signals from multiple activity devices (one of which being the activity device 102) and may receive signals from multiple healthcare establishment devices (one of which being the healthcare establishment device 106). The signal association component 118 may use the signals received from the multiple activity devices and the multiple healthcare establishment devices to determine which patient to associate with which healthcare establishment device. For instance, the signal association component 118 may receive multiple QRS complex signals from multiple activity devices being worn by different patients, and associate the patient 104 with the healthcare establishment device 106 based on a QRS signal from the activity device 102 most closely aligning with a cardiac signal received from the healthcare establishment device 106.

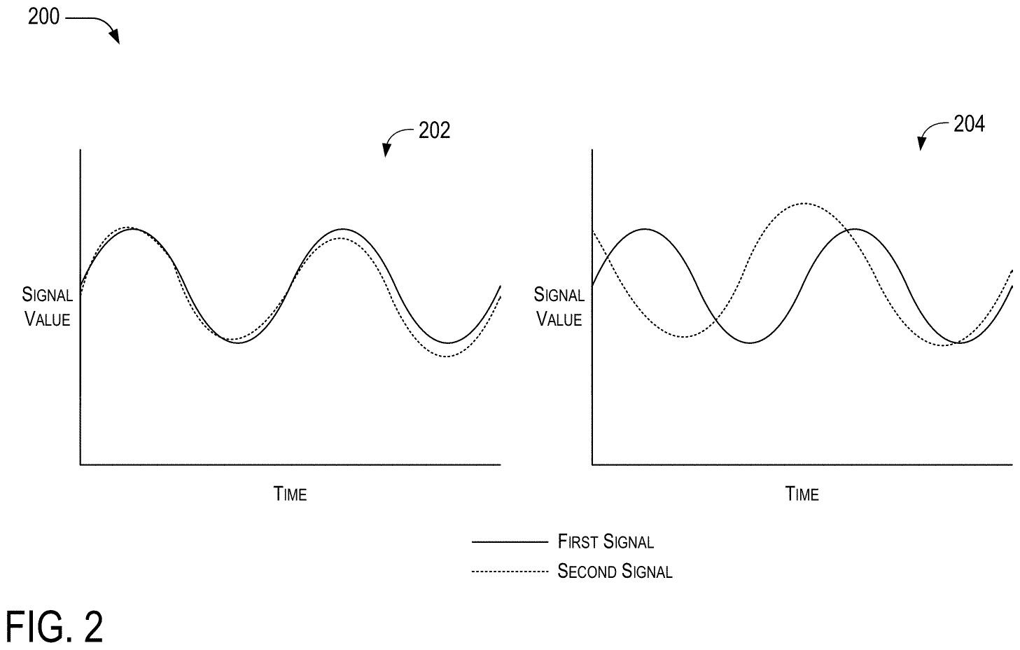

Example configurations of the activity device 102, the healthcare establishment device 106, and/or the clinician device 108, and methods for their use, are shown and described with reference to at least FIGS. 2-6 below.

FIG. 2 shows an example 200 of signals which may be used to determine whether to associate a patient with a healthcare establishment device.

For example, a first diagram 202 illustrates signal values on an x-axis of the diagram versus time on a y-axis of the diagram. The first diagram 202 includes a first signal (e.g., represented by a solid line) and a second signal (e.g., represented by a dotted line). In some examples, the activity device 102 of FIG. 1 may generate and output the first signal, and the healthcare establishment device 106 may generate and output the second signal. The first signal and/or the second signal may correspond to a measured physiological parameter of the patient 104, such as movement, heart rate, respiratory rate, blood pressure, SpO₂, and so forth. The signal association component 118 may receive the first signal and the second signal illustrated in the diagram 202, and determine, based at least in part on a temporal component of the first signal and of the second signal, whether both signals correspond to the patient 104.

In some examples, the signal association component 118 may determine a phase shift for one (or both) of the first signal and the second signal, where the phase shift may be based on a transmission delay of the first signal and/or the second signal. The signal association component 118 may apply a phase shift to the first signal and/or the second signal based on the transmission delay such that signal values occurring at a same (or similar) time are at a same (or similar) location along the time axis (the y-axis). Alternatively or additionally, the signal association component 118 may determine a period for each of the first signal and the second signal, where the period corresponds to an amount of time between peaks (and/or troughs) of the respective signals. If the period associated with the first signal is within a threshold amount of the period associated with the second signal, the signal association component 118 may determine that the first signal and the second signal are associated with the same patient 104. In some instances, the signal association component 118 may determine the period after a phase shift has been applied to the first signal and/or the second signal, such that the signal association component 118 measures periods occurring generally at a same time.

In some cases, the signal association component 118 may determine an amplitude of the first signal and/or the second signal, where the amplitude corresponds to a height from a lowest point of the signal to a highest point of the signal, and may be divided by two. In examples, the signal association component 118 may determine the amplitude after a phase shift has been applied to the first signal and/or the second signal, such that the signal association component 118 measures amplitudes occurring generally at a same time. The signal association component 118 may determine whether an amplitude of the first signal is within a threshold amount of an amplitude of the second signal at a same or similar time. If the amplitude associated with the first signal is within the threshold amount of the amplitude of the second signal at a same or similar time, the signal association component 118 may determine that the first signal and the second signal are associated with the same patient 104.

The signal association component 118 may use one and/or both of the period and the amplitude of the first signal and the second signal as factors to determine whether to associate the patient 104 with the healthcare establishment device 106. For instance, the signal association component 118 may use both the period and the amplitude of respiratory signals measured by the activity device 102 and the healthcare establishment device 106 to determine whether to associate the patient 104 with the healthcare establishment device 106. However, the signal association component 118 may rely on the amplitude without the period when comparing movement signals measured by the activity device 102 and the healthcare establishment device 106 to determine whether to associate the patient 104 with the healthcare establishment device 106, as movement signals may be more irregular than respiratory (and other types of) signals.

The first diagram 202 illustrates that the first signal and the second signal have a generally similar period and amplitude, where values of the first signal are similar to values of the second signal at similar times. In this case, the signal association component 118 may associate the patient 104 wearing the activity device 102 with the healthcare establishment device 106 that output the second signal. Although the illustrated signal values in the first diagram are not exactly the same in some instances, the signal association component 118 may determine that the signals are within a threshold amount of one another for a minimum about of time in making the determination to associate the patient 104 with the healthcare establishment device 106.

In an illustrative example, the activity device 102 and the healthcare establishment device 106 may each detect an event having a relatively short duration, such as a cardiac pulse or a QRS complex. Sensors associated with the activity device 102 and/or the healthcare establishment device 106 may output indications of such events as a binary or other type of output (e.g., 1 or 0) occurring at a specific time. In some examples, the signal association component 118 may apply a time window relative to the event, in which the signal association component 118 will associate the patient 104 with the healthcare establishment device 106 even if the timestamps associated with the respective events do not exactly match. For example, the signal association component 118 may apply time windows corresponding to a triangle pulse (e.g., 50 milliseconds, 100 milliseconds, 200 milliseconds, etc.), a trapezoid pulse (e.g., 50 milliseconds, 100 milliseconds, 200 milliseconds, etc.), a Gaussian, or other type of application to the event timestamp, which in some cases may be centered on the event timestamp, applied before or after the event timestamp, and so forth. The signal association component 118 may apply such time windows proximate or surrounding the events to one or both of the signals received from the activity device 102 or the healthcare establishment device 106. If the time window(s) applied to the event(s) at least partially overlap with one another, the signal association component 118 may associate the patient 104 with the healthcare establishment device 106.

A second diagram 204 also includes a first signal (e.g., represented by a solid line) and a second signal (e.g., represented by a dotted line). Similar to the first diagram 202 described above, the activity device 102 of FIG. 1 may generate and output the first signal, and the healthcare establishment device 106 may generate and output the second signal. The first signal and/or the second signal may correspond to a measured physiological parameter of the patient 104, such as movement, heart rate, respiratory rate, blood pressure, SpO₂, and so forth. The signal association component 118 may receive the first signal and the second signal illustrated in the second diagram 204, and determine, based at least in part on a temporal component of the first signal and of the second signal, whether both signals correspond to the patient 104. The signal association component 118 may determine and apply a phase shift for the first signal and/or the second signal, determine and compare periods for the first signal and the second signal, and/or determine and compare amplitudes of the first signal and the second signal at same or similar times, as described above.

The second diagram 202 illustrates that the first signal and the second signal do not have a generally similar period or amplitude values of the first signal and the second signal at similar times. In this case, the signal association component 118 may prevent the patient 104 wearing the activity device 102 from being associated with the healthcare establishment device 106 that output the second signal. Although the illustrated signal values in the first diagram are similar to one another at a few times, the signal association component 118 may determine that the signals are not within a threshold amount of one another for at least a minimum about of time in making the determination to prevent the patient 104 from being associated with the healthcare establishment device 106.

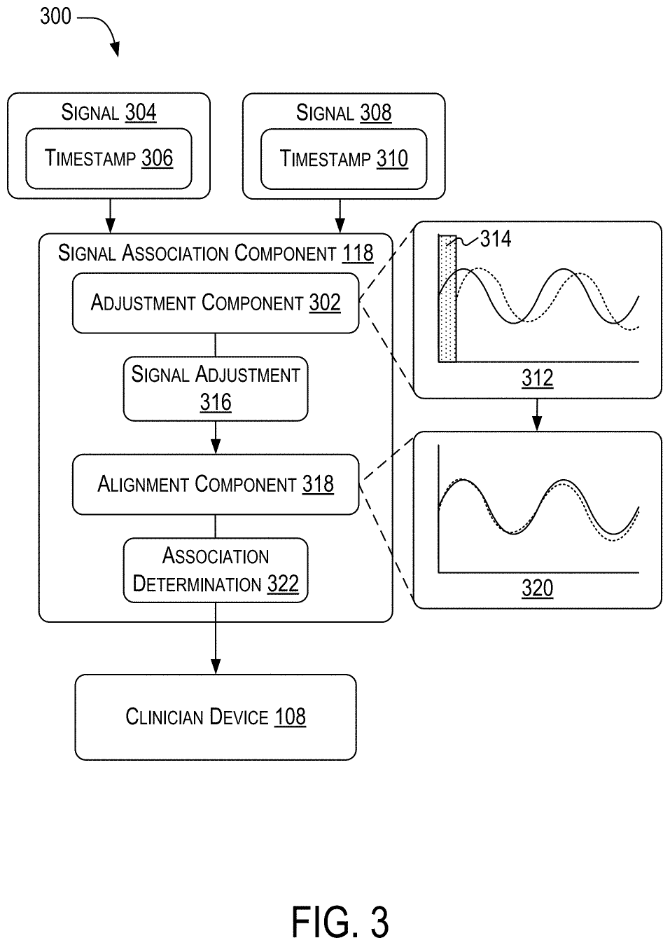

FIG. 3 shows an example system 300 configured to adjust and/or align signals based on a delay associated with at least one of the signals.

In some examples, the signal association component 118 of FIG. 1 may include an adjustment component 302 configured to adjust one or more signals due to transmission delay (or other type of delay). For instance, the adjustment component 302 may receive a signal 304 that includes a timestamp 306, and receive a signal 308 that includes a timestamp 310. In some cases, the activity device 102 may output the signal 304 to the signal association component 118 while the healthcare establishment device 106 outputs the signal 308 to the signal association component 118, as described in relation to FIG. 1. The timestamp 306 may be associated with a value included in the signal 304, where the value corresponds to a physiological parameter of the patient 104 as monitored by the activity device 104. The timestamp 310 may be associated with a value included in the signal 308, where the value corresponds to a physiological parameter as monitored by the healthcare establishment device 106.

The adjustment component 302 may use the timestamp 306 and the timestamp 310 to determine a phase shift to apply to one or both of the signals 304 and 308, such that a value associated with the timestamp 306 aligns with a value associated with the timestamp 310. For instance, a diagram 312 illustrates the signal 304 (corresponding to the solid line) and the signal 308 (corresponding to the dotted line). A shaded area 314 represents a transmission delay of the signal 308, as determined by the adjustment component 302 based on the timestamp 306 and the timestamp 310. The adjustment component 302 may determine a phase shift to apply to the signal 308 based on the transmission delay and represented by the shaded area 314.

In some cases, even using the timestamps 306 and 310, there may be an uncertainty associated with the transmission delay. For example, a timekeeping component of the activity device 102 may not be exactly aligned with a timekeeping component of the healthcare establishment device 106. Alternatively or additionally, the activity device 102 may collect values included in the signal 304 at a different rate than the values collected by the healthcare establishment device 106 and included in the signal 308, causing events to be recorded at different times according to the different collection rates. The adjustment component 302 may output a signal adjustment 316 to apply to the signal 308 based on the phase shift to an alignment component 318.

The adjustment component 302 may take uncertainties into account when determining whether and how to shift the signal 304 and/or the signal 308. For instance, the adjustment component 302 may determine an uncertainty associated with a value included in the signal 304, and/or an uncertainty associated with a value included in the signal 308. If the uncertainty associated with a value included in the signal 304 and/or the uncertainty associated with a value included in the signal 308 are less than a threshold uncertainty, then the adjustment component 302 may output the signal adjustment 316. Otherwise, the adjustment component 302 may prevent the signal adjustment 316 from being output, thus preventing the patient 104 from being associated with the healthcare establishment device 106 in some cases.

In an illustrative example, the adjustment component 302 may apply a threshold uncertainty of 100 milliseconds for cardiac pulses detected in the signal 304 and the signal 308. If a value associated with a cardiac pulse included in the signal 304, and/or a value associated with a cardiac pulse included in the signal 308 has an uncertainty of less than 100 milliseconds, the adjustment component may output the signal adjustment 316. However, if a value associated with a cardiac pulse included in the signal 304, and/or a value associated with a cardiac pulse included in the signal 308 has an uncertainty equal to or more than 100 milliseconds, the adjustment component may prevent the signal adjustment 316 from being output.

The alignment component 318 may be configured to align the signal 304 with the signal 308 based at least in part on the signal adjustment 316. Additionally, in some cases, the alignment component 318 may determine whether the signal 304 aligns with the signal 308 after the phase shift represented by the signal adjustment 316 is applied. For example, the alignment component 318 may determine whether an amplitude of the signal 304 is within a threshold amount of an amplitude of the signal 308. Alternatively or additionally, the alignment component 318 may determine whether an period of the signal 304 is within a threshold amount of a period of the signal 308.

The alignment component 318 may use different thresholds for different signal types. In some cases, the alignment component 318 may use a threshold amount of 1%, 5%, 10%, or some other percentage of a value of the signal 304 or the signal 308 for an amplitude threshold, and/or may use a threshold amount of 1%, 5%, 10%, or some other percentage of a period duration of the signal 304 or the signal 308 for period threshold. In some examples, the alignment component 318 may determine whether the signals 304 and 308 align within a threshold amount of the amplitude and/or period for at least a minimum amount of time (e.g., greater than 50%, 75%, 90%, 99% of 5 measured seconds, 10 measured seconds, 1 measured minute, etc.). If the signal 304 aligns with the signal 308 (e.g., within a threshold amount for at least a minimum amount of time), the alignment component 318 may output an association determination 322 to the clinician device 108 as described above, where the association determination indicates that the patient 104 is associated with the healthcare establishment device 106.

In examples, the signal association component 118 may be configured to associate the patient 104 with the healthcare establishment device 106 using different signal types and/or based on different physiological parameters. For instance, the adjustment component 302 may determine that the signal 304 is a different signal type than the signal 308 (e.g., the signal 304 is a movement signal and the signal 308 is a pressure signal). The adjustment component 302 may detect an event included in the signal 304, such as a change in posture of the patient 104 indicated in the signal 304 generated by the activity device 102. Additionally, the adjustment component 302 may detect an event included in the signal 308, such as a weight transfer from a laying position to a sitting position on the healthcare establishment device 106 that generates the signal 308. In another example, the activity device 102 may output the signal 304 indicating a QRS complex of the patient 104, and the healthcare establishment device 106 may output the signal 308 indicating a cardiac pulse detected by pressure sensors in a hospital bed. In some cases, a QRS complex and a cardiac pulse may have different morphologies in the respective signals 304 and 308, and therefore the timestamp 306 may not directly correlate with the timestamp 310.

Therefore, the adjustment component 302 may cross-correlate the event identified in the signal 304 with the event identified in the signal 308. The adjustment component 302 may determine a difference in time of the event indicated in the signal 302 and the event indicated in the signal 308, and if the difference in time is less than a threshold difference, the alignment component 318 may determine that the signal 304 aligns with the signal 308 and output the association determination 322 accordingly. Alternatively or additionally, the adjustment component 302 may associate the events detected in the signals 304 and 308 with time windows at or near the timestamp 306 and/or the timestamp 310. For example the adjustment component 302 may replace the timestamp 306 and/or the timestamp 310 with a fixed shape pulse, such as a triangle pulse (e.g., 50 milliseconds, 100 milliseconds, 200 milliseconds, etc.), a trapezoid pulse (e.g., 50 milliseconds, 100 milliseconds, 200 milliseconds, etc.), a Gaussian pulse, and the like, which may be centered on the timestamp 306 and/or the timestamp 310. The adjustment component 302 may replace the remainder of the signal 304 and/or the signal 308 (e.g., at locations other than the entered pulse(s)) with indications of no event, such as by a “zero” value. The alignment component 318 may correlate sequences of pulses corresponding to events of different types over time to determine whether to associate the patient 104 with the healthcare establishment device 106.

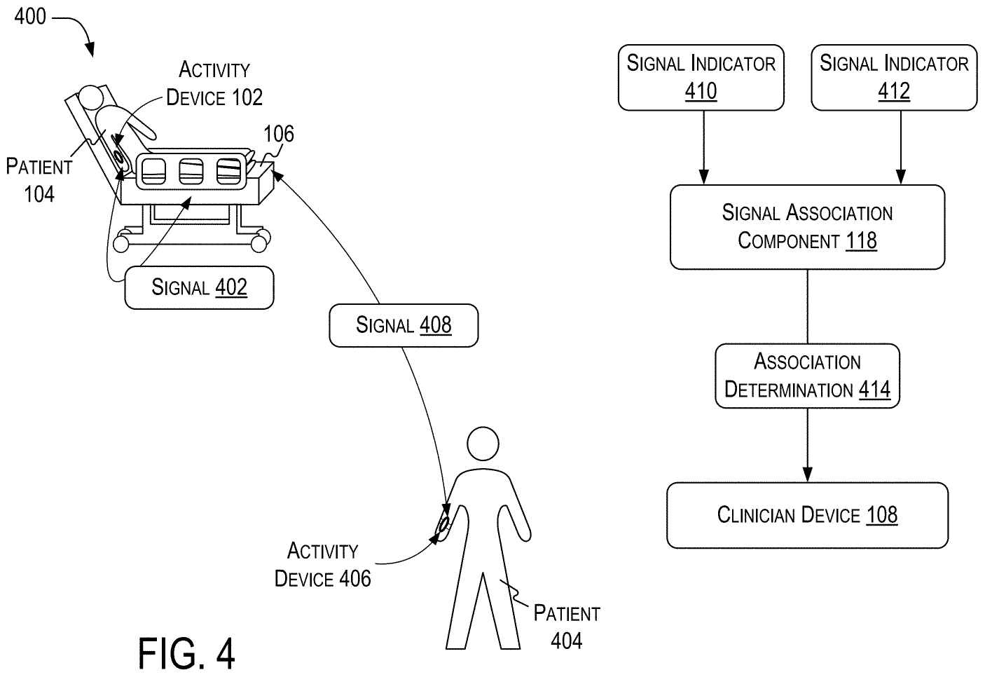

FIG. 4 shows a schematic block diagram 400 of a signal association component that may utilize signal strength of signals in determining whether to associate a patient with a healthcare establishment device.

In some examples, the block diagram 400 includes the activity device 102, the patient 104, and the healthcare establishment device 106, which may have similar features as described in relation to FIG. 1. The activity device 102 may output and/or receive a signal 402 with the healthcare establishment device 106, which may include information such as patient data associated with the patient 104 and/or a signal indicator indicating a strength of the signal, signal type of the signal, and the like. The signal 402 may be a Bluetooth signal, a wireless signal, an RF signal, or other signal type. The signal strength of the signal 402 may be based on a distance between the activity device 102 and the healthcare establishment device 106, whether an object is blocking a path of the signal 402, and so forth. Additionally, the block diagram 400 may include a patient 404 wearing an activity device 406, which may have similar or different functionality as the activity device 102. The activity device 406 may output and/or receive a signal 408 with the healthcare establishment device 106, which may include information such as patient data associated with the patient 404 and/or a signal indicator indicating a strength of the signal, signal type of the signal, and the like. The signal 406 may be a Bluetooth signal, a wireless signal, an RF signal, or other signal type. The signal strength of the signal 408 may also be based on a distance between the activity device 406 and the healthcare establishment device 106, whether an object is blocking a path of the signal 408, and so forth.

In examples, the activity device 102 and/or the healthcare establishment device 106 may output a signal indicator 410 to the signal association component 118 described in relation to FIG. 1. The signal indicator 410 may represent a signal strength of the signal 402 communicated between the activity device 102 and the healthcare establishment device 106. Additionally, in some cases, the activity device 406 and/or the healthcare establishment device 106 may output a signal indicator 412 to the signal association component 118. The signal indicator 412 may represent a signal strength of the signal 408 communicated between the activity device 406 and the healthcare establishment device 106.

The signal association component 118 may compare the signal strength represented by the signal indicator 410 with the signal strength represented by the signal indicator 412 to determine which signal strength is greater. The signal association component 118 may use the signal strength comparison as a factor in determining whether to associate the patient 104 or the patient 404 with the healthcare establishment device 106. For instance, the signal association component 118 may use the signal strength factor in addition to factors associated with whether signals generated by the activity devices 102 and 406 align with signals generated by the healthcare establishment device 106. In some cases, the signal association component 118 may rely on the signal strength factor without other factors, such as when signals generated by the activity devices 102 and 406 or by the healthcare establishment device 106 are inconclusive as to whether the signals align with one another. In some examples, the signal association component 118 may output an association determination 414 to the clinician device 108 as described above. The association determination 414 may be based on the signal strength in combination with alignment of signals generated by the activity device 102 (or the activity device 406) and the healthcare establishment device 106, or may be independent of signals generated by the activity device 102 (or the activity device 406) and the healthcare establishment device 106.

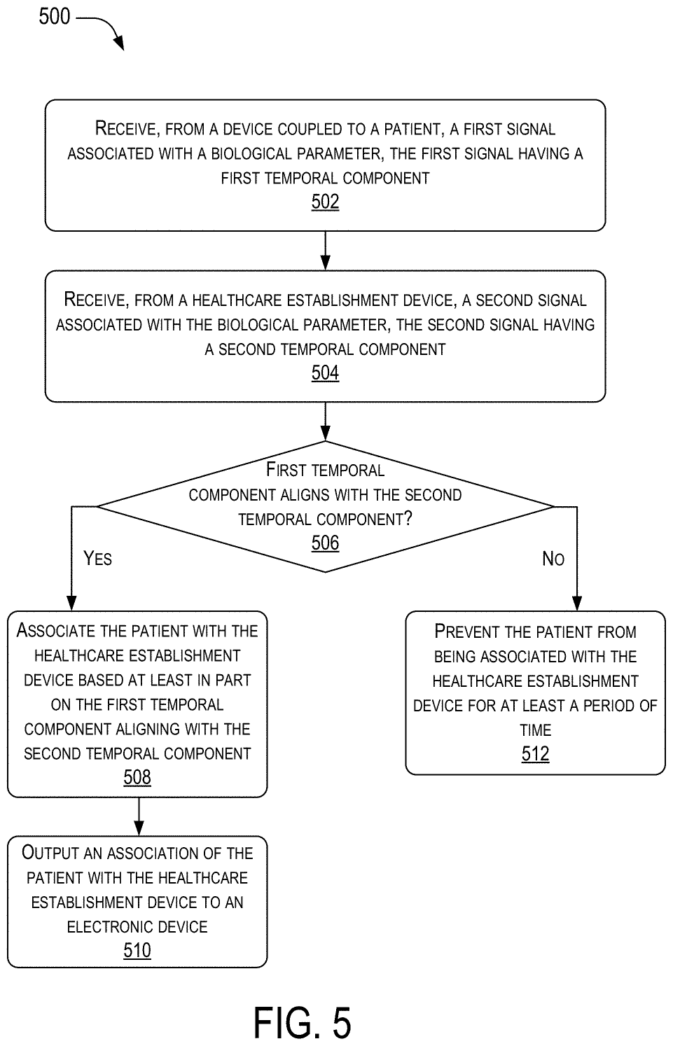

FIG. 5 is an example process 500 for associating a patient with a healthcare establishment device based on whether signals that may be associated with the patient at least partially align, according to the techniques described herein. In some examples, the process 500 may be performed by one or more processors of computing devices, such as the patient management system of FIG. 1.

At operation 502, the process can include receiving, by one or more processors of a patient management system and from a device coupled to a patient, a first signal associated with a physiological parameter, where the first signal has a first temporal component. For instance, the patient management system 110 may receive a signal associated with a physiological parameter from the activity device 102 worn by the patient 104. The physiological parameter may include, but is not limited to, movement, heart rate, respiratory rate, blood pressure, SpO₂, and so forth. The temporal component may, in some cases, be associated with a time that a value of the physiological parameter was collected, sensed, and/or generated by the activity device 102.

At operation 504, the process can include receiving, by the one or more processors and from a healthcare establishment device, a second signal associated with the physiological parameter, the second signal having a second temporal component. In examples, the patient management system 110 may receive a signal associated with a physiological parameter from the healthcare establishment device 106. The physiological parameter may include, but is not limited to, movement, heart rate, respiratory rate, blood pressure, SpO₂, and so forth. The physiological parameter may be a same physiological parameter as represented by the signal received from the activity device 102, and/or may be a different physiological parameter. The temporal component may, in some cases, be associated with a time that a value of the physiological parameter was collected, sensed, and/or generated by the healthcare establishment device 106.

At operation 506, the process can include determining, by the one or more processors, whether the first temporal component aligns with the second temporal component. For example, the alignment component 302 may apply a phase shift to the first signal and/or the second signal based on timestamps included in the respective signals to account for transmission delay(s) of the signal(s). In some cases, the adjustment component 318 may determine whether the signals align based on comparing periods and/or amplitudes of the respective signals to each other.

If the periods and/or amplitudes are within a threshold amount of one another at a same or similar time, the adjustment component 318 may determine that the signals align (e.g., “Yes” at operation 506), and the process may proceed to operation 508. The operation 508 may include associating, by the one or more processors, the patient with the healthcare establishment device based at least in part on the first temporal component aligning with the second temporal component. For example, the signal association component 118 may determine that the patient 104 is associated with the healthcare establishment device 106. In some cases, the process may include outputting, by the one or more processors, an association of the patient with the healthcare establishment device to an electronic device. For instance, the patient management system 110 may output a verification message to the clinician device 108, which allows a clinician to verify that the patient 104 is associated with the healthcare establishment device 106. Alternatively or additionally, the patient management system 110 may output the association to the activity device 102 and/or the healthcare establishment device 106, which may enable one or more of these devices to track subsequent activities of the patient 104 more accurately.

If the periods and/or amplitudes are not within a threshold amount of one another at a same or similar time, the adjustment component 318 may determine that the signals do not align (e.g., “No” at operation 506), and the process may proceed to operation 512. The operation 512 may include preventing, by the one or more processors, the patient from being associated with the healthcare establishment device for at least a period of time. For example, the patient 104 may be prevented from being associated with the healthcare establishment device for 10 minutes, one hour, one day, or the like if the signals do not align as described above. However, the process 500 may be repeated at a subsequent time (e.g., after the period of time that the patient 104 is prevented from being associated with the healthcare establishment device 106) to account for the patient 104 switching hospital rooms or beds, to name a few examples.

### Example System and Device

FIG. 6 illustrates an example system generally at 600 that includes an example computing device 602 that is representative of one or more computing systems and/or devices that may implement the various techniques described herein. This is illustrated through inclusion of the patient management system 110. The computing device 602 may be, for example, a server of a service provider, a device associated with a client (e.g., a client device), an on-chip system, and/or any other suitable computing device or computing system.

The example computing device 602 as illustrated includes a processing system 604, one or more computer-readable media 606, and one or more I/O interface 608 that are communicatively coupled, one to another. Although not shown, the computing device 602 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 604 is representative of functionality to perform one or more operations using hardware. Accordingly, the processing system 604 is illustrated as including hardware element 610 that may be configured as processors, functional blocks, and so forth. This may include implementation in hardware as an application specific integrated circuit or other logic device formed using one or more semiconductors. The hardware elements 610 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable storage media 606 is illustrated as including memory/storage 612. The memory/storage 612 represents memory/storage capacity associated with one or more computer-readable media. The memory/storage component 612 may include volatile media (such as random access memory (RAM)) and/or nonvolatile media (such as read only memory (ROM), Flash memory, optical disks, magnetic disks, and so forth). The memory/storage component 612 may include fixed media (e.g., RAM, ROM, a fixed hard drive, and so on) as well as removable media (e.g., Flash memory, a removable hard drive, an optical disc, and so forth). The computer-readable media 606 may be configured in a variety of other ways as further described below.

Input/output interface(s) 608 are representative of functionality to allow a user to enter commands and information to computing device 602, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 602 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” “logic,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on and/or transmitted across some form of computer-readable media. The computer-readable media may include a variety of media that may be accessed by the computing device 602. By way of example, and not limitation, computer-readable media may include “computer-readable storage media” and “computer-readable transmission media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer-readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable transmission media” may refer to a medium that is configured to transmit instructions to the hardware of the computing device 602, such as via a network. Computer-readable transmission media typically may transmit computer-readable instructions, data structures, program modules, or other data in a modulated data signal, such as carrier waves, data signals, or other transport mechanism. Computer-readable transmission media also include any information delivery media. The term “modulated data signal” means a signal that has one or more of its characteristics set or changed in such a manner as to encode information in the signal. By way of example, and not limitation, computer-readable transmission media include wired media such as a wired network or direct-wired connection, and wireless media such as acoustic, radio frequency (RF), infrared, and other wireless media.

As previously described, hardware elements 610 and computer-readable media 606 are representative of modules, programmable device logic and/or device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 610. The computing device 602 may be configured to implement particular instructions and/or functions corresponding to the software and/or hardware modules. Accordingly, implementation of a module that is executable by the computing device 602 as software may be achieved at least partially in hardware, e.g., through use of computer-readable storage media and/or hardware elements 610 of the processing system 604. The instructions and/or functions may be executable/operable by one or more articles of manufacture (for example, one or more computing devices 602 and/or processing systems 604) to implement techniques, modules, and examples described herein.

The techniques described herein may be supported by various configurations of the computing device 602 and are not limited to the specific examples of the techniques described herein. This functionality may also be implemented all or in part through use of a distributed system, such as over a “cloud” 614 via a platform 616 as described below.

The cloud 614 includes and/or is representative of a platform 616 for resources 618. The platform 616 abstracts underlying functionality of hardware (e.g., servers) and software resources of the cloud 614. The resources 618 may include applications and/or data that can be utilized while computer processing is executed on servers that are remote from the computing device 602. Resources 618 can also include services provided over the Internet and/or through a subscriber network, such as a cellular or Wi-Fi network.

The platform 616 may abstract resources and functions to connect the computing device 602 with other computing devices. The platform 616 may also be scalable to provide a corresponding level of scale to encountered demand for the resources 618 that are implemented via the platform 616. Accordingly, in an interconnected device embodiment, implementation of functionality described herein may be distributed throughout multiple devices of the system 600. For example, the functionality may be implemented in part on the computing device 602 as well as via the platform 616 which may represent a cloud computing environment 614.

The example systems and methods of the present disclosure overcome various deficiencies of known prior art devices. Other embodiments of the present disclosure will be apparent to those skilled in the art from consideration of the specification and practice of the disclosure contained herein. It is intended that the specification and examples be considered as example only, with a true scope and spirit of the present disclosure being indicated by the following claims.

What is claimed is:

1. A system comprising:
one or more processors; and
one or more computer-readable media storing instructions that, when executed by the one or more processors, cause the one or more processors to perform operations comprising:
receiving, from a device coupled to a patient, a first signal associated with a physiological parameter, the first signal having a first temporal component;
receiving, from a healthcare establishment device, a second signal associated with the physiological parameter, the second signal having a second temporal component;
determining that the first temporal component aligns with the second temporal component; and
associating the patient with the healthcare establishment device based at least in part on the first temporal component aligning with the second temporal component.

2. The system of claim 1, the operations further comprising determining that the second signal corresponds to the physiological parameter of the patient based at least in part on the first temporal component aligning with the second temporal component.

3. The system of claim 1, wherein the healthcare establishment device is a hospital bed, the hospital bed comprising a load cell, a capacitive sensor, or a pressure sensor, and
wherein the second signal received from the hospital bed comprises a motion signal, a respiration signal, a ballistocardiogram signal, or a cardiac pulse signal generated by the load cell, the capacitive sensor, or the pressure sensor.

4. The system of claim 1, wherein the first signal comprises:
an electrocardiogram (ECG) signal;
a respiration signal;
an impedance cardiogram signal;
a photoplethysmogram (PPG) signal;
an ultrasound signal; or
a motion signal.

5. The system of claim 1, the operations further comprising determining a first transmission delay associated with the first signal and a second transmission delay associated with the second signal,
wherein determining that the first temporal component aligns with the second temporal component is based at least in part on the first transmission delay and the second transmission delay.

6. The system of claim 1, wherein the first signal indicates a change in posture of the patient and the second signal indicates a pressure associated with a weight of the patient.

7. The system of claim 1, wherein the physiological parameter is a cardiac pulse or a breath of the patient.

8. The system of claim 1, wherein the device is a first device, the operations further comprising:
determining a first signal strength of a third signal, the third signal communicating first data from the first device to the healthcare establishment device;
determining a second signal strength of a fourth signal, the fourth signal communicating second data from a second device to the healthcare establishment device; and
determining that the first signal strength is greater than the second signal strength,
wherein associating the patient with the healthcare establishment device is further based on the first signal strength being greater than the second signal strength.

9. The system of claim 1, the operations further comprising:
determining a first uncertainty associated with the first temporal component;
determining a second uncertainty associated with the second temporal component; and
determining that the first uncertainty or the second uncertainty are less than a threshold uncertainty,
wherein determining that the first temporal component aligns with the second temporal component is based at least in part on the first uncertainty or the second uncertainty being less than the threshold uncertainty.

10. The system of claim 1, the operations further comprising:
determining that the first signal is a different signal type than the second signal;
detecting a first event included in the first signal and a second event included in the second signal based at least in part on the first signal being the different signal type than the second signal; and
cross-correlating the first event and the second event to determine an association of the first event and the second event,
wherein determining that the first temporal component aligns with the second temporal component is based at least in part on the association.

11. The system of claim 10, the operations further comprising determining that a difference between a first time associated with the first event and a second time associated with the second event is less than a threshold difference,
wherein determining that the first temporal component aligns with the second temporal component is further based on the difference being less than the threshold difference.

12. A method comprising:
receiving, from a first device coupled to a patient, a first signal associated with a physiological parameter, the first signal having a first temporal component;
receiving, from a second device, a second signal associated with the physiological parameter, the second signal having a second temporal component;
determining that the first temporal component aligns with the second temporal component; and
associating the patient with the second device based at least in part on the first temporal component aligning with the second temporal component.

13. The method of claim 12, further comprising determining that the second signal corresponds to the physiological parameter of the patient based at least in part on the first temporal component aligning with the second temporal component.

14. The method of claim 12, further comprising determining a first transmission delay associated with the first signal and a second transmission delay associated with the second signal,
wherein determining that the first temporal component aligns with the second temporal component is based at least in part on the first transmission delay and the second transmission delay.

15. The method of claim 12, further comprising:
determining a first signal strength of a third signal, the third signal communicating first data from the first device to the second device;
determining a second signal strength of a fourth signal, the fourth signal communicating second data from a third device to the second establishment device; and
determining that the first signal strength is greater than the second signal strength,
wherein associating the patient with the second device is further based on the first signal strength being greater than the second signal strength.

16. The method of claim 12, further comprising:
determining that the first signal is a different signal type than the second signal;
detecting a first event included in the first signal and a second event included in the second signal based at least in part on the first signal being the different signal type than the second signal; and
cross-correlating the first event and the second event to determine an association of the first event and the second event,
wherein determining that the first temporal component aligns with the second temporal component is based at least in part on the association.

17. One or more computer-readable media storing instructions that, when executed by one or more processors, cause the one or more processors to perform operations comprising:
receiving, from a first device coupled to a patient, a first signal associated with a physiological parameter, the first signal having a first temporal component;
receiving, from a second device, a second signal associated with the physiological parameter, the second signal having a second temporal component;
determining that the first temporal component aligns with the second temporal component; and
associating the patient with the second device based at least in part on the first temporal component aligning with the second temporal component.

18. The one or more computer-readable media of claim 17, wherein the first signal comprises:
an electrocardiogram (ECG) signal;
a respiration signal;
an impedance cardiogram signal;
a photoplethysmogram (PPG) signal;
an ultrasound signal; or
a motion signal.

19. The one or more computer-readable media of claim 17, wherein the second device is a hospital bed, the hospital bed comprising a load cell a capacitive sensor, or a pressure sensor, and
wherein the second signal received from the hospital bed comprises a motion signal, a respiration signal, a ballistocardiogram signal, or a cardiac pulse signal generated by the load cell, the capacitive sensor, or the pressure sensor.

20. The one or more computer-readable media of claim 17, wherein the first signal indicates a change in posture of the patient and the second signal indicates a pressure associated with a weight of the patient.
